(19) United States
(12) Patent Application Publication
(10) Pub. No.: US 2002/0192307 A1
(43) Pub. Date: Dec. 19, 2002

(54) Method and composition for enhancing sexual desire

(76) Inventors: Hank C.K. Wuh, Los Altos, CA (US); Aileen S. Trant, Mountain View, CA (US)

Correspondence Address: James C. Wray, Suite 300, 1493 Chain Bridge Road, McLean, VA, 22101, US

(21) Appl. No.: 10/157,185

(22) Filed: May 30, 2002

Related U.S. Application Data
(60) Provisional application No. 60/295,297, filed on Jun. 1, 2001.

(51) Int. Cl. A61K 35/78; A61K 31/198

(52) U.S. Cl. 424/728; 424/725; 424/752; 514/565

(57) ABSTRACT

The dietary supplement for wellness includes in a combination ofL-arginine, ginseng, ginkgo biloba, and damiana. The unit dosage is in a capsule or a tablet form or as an aqueous composition. The unit dosage may include an amount of antioxidant vitamins, vitamin B complex, and minerals sufficient to provide up to about 100% to about 200% of the percent daily values of the antioxidant vitamins, vitamin B complex, and minerals upon administering an appropriate quantity of the unit dosages to achieve a desired daily dosage. The daily dosage may be in an amount of up to about 600 ml. of an aqueous composition and may be administered as a beverage. The composition may be administered daily to a subject for a time sufficient for sexually arousing the subject more easily than without administration of the combination. The daily dosage is administered to the subject for a time sufficient to improve the quality of orgasm for the subject and/or to improve the frequency of desire to engage in sexual activity.

This application claims the benefit of U.S. Provisional Application No. 60/295,297, filed Jun. 1, 2001.

### BACKGROUND OF THE INVENTION

This invention relates to the maintenance of a state of wellness in which sexual health is improved.

Quality of life is increasingly valued in today's society. Proper nutrition and exercise, and healthy sexual function contribute to maintain an overall state of well being, which can serve to manage stress, maintain a properly functioning immune system, protect against disease, maintain a positive mental outlook, and generally to enable one to feel good and enjoy life.

### SUMMARY OF THE INVENTION

The present invention incorporates by reference the disclosure of co-pending application Ser. No. 09/410,446, now patent 6,368,640, in its entirety, which describes the nutritional supplement ArginMax.

During a study on ArginMax for women, the daily nutritional supplement designed to improve sexual fitness and health, an unexpected finding was made. Namely, it was discovered that ArginMax was able to significantly increase libido in women participating in the study versus those on placebo. Specifically, both the level of sexual desire, and the frequency of sexual desire were enhanced significantly in the ArginMax group.

Due to the fact that libido is regulated centrally, and involves the CNS, there is an inventive aspect that is both unexpected and novel. Certainly, this is a separate phenomenon from the nitric oxide pathway that ArginMax was originally formulated to enhance as described in the co-pending application incorporated herein by reference. In the present application ArginMax is used to enhance libido and arousal.

Specifically, ArginMax combines certain ingredients such as I-arginine, ginkgo, and ginseng because of their ability to synergistically enhance nitric-oxide production. Enhancing nitric oxide production (or inhibiting the breakdown of biochemical products for which nitric oxide triggers the production of) in the genitalia is known to enhance sexual functioning. In women, this would mean enhanced engorgement and lubrication. However, this functional mechanism is separate from that which regulates libido and arousal, a phenomenon of the central nervous system (CNS) perhaps modulated by sex hormones and specific receptors. It is apparent from our recent studies that ArginMax demonstrates significant ability to enhance libido and arousal in women in addition to enhancing sexual functioning via the nitric oxide cycle.

Given that other major reproductive functions, such as lactation and ovulation, both involve central feed back loop via the CNS, the present ArginMax formulation and the individual components contained in ArginMax may play an important role in the regulation and the optimization of CNS regulated libido. ArginMax for female sexual fitness is a nutritional supplement that incorporates a standardized combination of L-arginine, Korean ginseng (Panax ginseng—30% ginsenosides), Ginkgo biloba (24% flavone glycosides, 6% terpene lactones), and damiana leaf (Turnera aphrodisiaca), along with vitamins, A, C, E, B6, B12, biotin, folate, niacin, pantothenic acid, riboflavin, thiamin, and minerals, calcium, iron and zinc.

These and further and other objects and features of the invention are apparent in the disclosure, which includes the above and ongoing written specification and the claims.

### DETAILED DESCRIPTION OF PREFERRED EMBODIMENTS

The following outlines the materials, methods and results of the study that supports the unexpected findings discussed above. The results provide evidence of increases in libido as reported in the following findings:

Materials:

In a preferred embodiment, the dietary supplement for wellness includes, but is not limited to, in a unit dosage a combination of (a) L-arginine, (b) ginseng, (c) ginkgo biloba, and (d) damiana. Preferably, the unit dosage is in a capsule or a tablet form or as an aqueous composition.

The dietary supplement preferably includes about 50 mg to about 5000 mg L-arginine; about 5 mg to about 600 mg ginseng; about 5 mg to about 300 mg ginkgo biloba, and about 0 mg to about 300 mg damiana. The preferred composition may also include in a unit dosage an amount of antioxidant vitamins, vitamin B complex, and minerals sufficient to provide up to about 100% to about 200% of the percent daily values of the antioxidant vitamins, vitamin B complex, and minerals upon administering an appropriate quantity of the unit dosages to achieve a desired daily dosage. The daily dosage may be in an amount of, but is not limited to, about 50 ml to about 600 ml. of an aqueous composition and may be administered as a beverage.

A preferred dietary supplement may be in the form of an aqueous drink of about 100 ml comprising (a) about 1500 mg L-arginine; (b) about 100 mg Korean ginseng; (c) about 100 mg ginkgo biloba; (d) about 50 mg damiana; (e) about 100% of the daily value of each of vitamins A, C, and E; (f) about 100% of the daily value of each of thiamin, riboflavin, niacin, vitamin B-6, folate, vitamin B-12, biotin, pantothenic acid; and (g) about 50% of the daily value of iron, calcium, and zinc. Additionally, the desired daily dosage of the dietary supplement may include administering up to 200% of the percent daily values of antioxidant vitamins and vitamin B complex, and up to 100% of the percent daily value of minerals.

The composition may be administered daily to a subject for a time sufficient for sexually arousing the subject more easily than without administration of the combination. The daily dosage is administered to the subject for a time sufficient to improve the quality of orgasm for the subject. The dietary supplement may be administered to the subject for a period of time sufficient to improve the frequency of desire to engage in sexual activity.

Methods:

All participants were enrolled based on their expressed interest in improving their current sexual function. Twelve had been previously treated for sexual dysfunction. Ninety-three (93) study participants, ages 22-73, successfully followed and completed the study protocol. The survey instrument was the Female Sexual Function Index (FSFI), a validated questionnaire with multidimensional scales for assessment of sexual function used in the evaluation of Viagra (sildenafil citrate) in women (Kaplan et al., 1999). Medical histories, including self-assessment of menopausal status, and FSFI scores were collected at 0 and 4 weeks. Upon enrollment, subjects were randomly assigned ArginMax or placebo in a double-blind fashion.

Of the 93 subjects who completed the test protocol, forty-six (46) subjects were on ArginMax and forty-seven (47) were on placebo. The age distributions were similar, with the ArginMax group ranging from 24 to 73 (mean 42.2), and the placebo group ranging from 22 to 68 (mean 41.3). Self-assessment of menopausal status resulted in 58 women being categorized as premenopausal (PRE), 16 as perimenopausal (PERI), and 19 as postmenopausal (POST).

Results:

Summaries of FSFI data in Table 1 demonstrate significant improvements in many parameters among the ArginMax (Active) groups as compared to the placebo groups, categorized as PRE, PERI or POST in menopausal status.

The PERI women on ArginMax reported the most improvements, as a group, including decreased dryness (Q2, 64% of group; p=0.07), increased frequency of sex (Q3, 91%, p<0.01), increased frequency of sexual desire (Q4, 73%), improved satisfaction with sexual relationship (Q7, 73%), and increased clitoral sensation (Q9, 73%). These same parameters increased only 20 to 40% in the PERI group on placebo.

PRE women on ArginMax improved primarily in desire (Q4 and 5, 58% vs 38-47% on placebo; p<0.05) and satisfaction with overall sex life (Q6, 67% vs. 35% on placebo; p<0.01). POST women, as a group, increased mainly in satisfaction with sexual relationship (Q7, 64% vs. 38% on placebo), and in level of sexual desire (Q5, 45% vs. 13% on placebo, p=0.08). These differences demonstrate a shift in the pattern of attributes that show improvement among the different menopausal status groups.

Overall (in Table 2), the largest improvements across all groups on ArginMax occurred in level of sexual desire (Q5, 64% vs. 43% on placebo, p<0.01), and in frequency of intercourse (Q3, 64% vs. 28% on placebo, p<0.001).

Combining PRE and PERI women on ArginMax resulted in 71% increase in sexual desire and 68% increase in satisfaction with overall sex life (vs. 49% and 36%, respectively, on placebo for Q5 and Q6; p<0.01).

Tables 1 and 2 demonstrate the significant differences in mean score increases for these parameters, the ArginMax group showing almost a full point increase, on average, more than the placebo group, in both sexual desire and satisfaction with overall sex life.

Since POST women increased mainly in satisfaction with sexual relationship, more so than in sex life satisfaction, combining groups dilutes the overall increase in percent improved for any one attribute. Additionally, significant increases (p<0.05) were noted in the combined ArginMax groups for degree of lubrication (Q2, decreased dryness), frequency of sexual intercourse (Q3) and sexual desire (Q4), satisfaction with sexual relationship (Q7), frequency of orgasm (Q8) and degree of clitoral sensation (Q9) relative to the placebo group (see Table 2).

No visual disturbance, blood pressure alterations, dizziness, hypersensitivity or other significant side effects were noted in either group. Four participants noted minor gastric disturbances, two noticed heavier bleeding during their periods and one had increased incidence of headaches.

Co-pending application, Ser. No. 09/410,446, now U.S. Pat. No. 6,368,640, which describes the nutritional supplement ArginMax, is herein incorporated by reference in its entirety.

While the invention has been described with reference to specific embodiments, modifications and variations of the invention may be constructed without departing from the scope of the invention, which is defined in the following claims.

TABLE 1
Results from the Female Sexual Function Index (FSFI), Active vs. Placebo

| | | | | | | | | | |
|---|---|---|---|---|---|---|---|---|---|
| FSFI Question | 1^{a} | 2^{b} | 3^{c} | 4^{d} | 5^{e} | 6^{f} | 7^{g} | 8^{h} | 9ⁱ |

Active, PRE: 24 women

| | | | | | | | | | |
|---|---|---|---|---|---|---|---|---|---|
| mean score, start | 3.8 | 3.5 | 2.9 | 2.2 | 1.9 | 2.0 | 2.5 | 3.0 | 2.9 |
| mean score 4 wks | 4.3 | 4.1 | 3.7 | 3.2 | 3.2 | 3.2 | 3.4 | 3.8 | 3.5 |
| % improved | 33% | 50% | 58% | 58% | 58% | 67% | 42% | 50% | 33% |

Placebo, PRE: 34 women

| | | | | | | | | | |
|---|---|---|---|---|---|---|---|---|---|
| mean score, start | 3.7 | 3.1 | 3.3 | 2.0 | 1.8 | 2.3 | 2.6 | 2.7 | 2.7 |
| mean score, 4 wks | 3.6 | 3.0 | 3.4 | 2.3 | 2.2 | 2.6 | 3.1 | 3.0 | 3.0 |
| % improved | 24% | 29% | 26% | 38% | 47% | 35% | 35% | 29% | 38% |
| p values | 0.28 | 0.13 | 0.004 | 0.04 | 0.01 | 0.004 | 0.15 | 0.13 | 0.21 |

Active, PERI: 11 women

| | | | | | | | | | |
|---|---|---|---|---|---|---|---|---|---|
| mean score, start | 3.6 | 2.8 | 3.4 | 2.1 | 1.6 | 2.0 | 2.5 | 2.5 | 2.6 |
| mean score, 4 wks | 4.2 | 3.7 | 4.6 | 2.9 | 2.3 | 3.1 | 3.6 | 3.4 | 3.6 |
| % improved | 36% | 64% | 91% | 73% | 64% | 64% | 73% | 55% | 73% |

Placebo, PERI 5 women

| | | | | | | | | | |
|---|---|---|---|---|---|---|---|---|---|
| mean score, start | 4.2 | 3.2 | 3.2 | 1.4 | 1.4 | 2.0 | 2.4 | 2.4 | 2.0 |
| mean score, 4 wks | 4.6 | 3.2 | 3.0 | 1.6 | 2.0 | 2.6 | 2.6 | 2.2 | 2.4 |
| % improved | 20% | 20% | 20% | 40% | 60% | 40% | 40% | 20% | 40% |
| p values | 0.31 | 0.07 | 0.004 | 0.11 | 0.43 | 0.20 | 0.11 | 0.12 | 0.11 |

Active, POST: 11 women

| | | | | | | | | | |
|---|---|---|---|---|---|---|---|---|---|
| mean score, start | 2.6 | 1.8 | 1.9 | 1.7 | .5 | 2.0 | 2.2 | 2.2 | 2.2 |
| mean score, 4 wks | 2.9 | 2.6 | 2.5 | 1.9 | 1.9 | 2.6 | 3.2 | 2.7 | 2.6 |
| % improved | 18% | 27% | 45% | 36% | 45% | 45% | 64% | 36% | 36% |

Placebo, POST: 8 women

| | | | | | | | | | |
|---|---|---|---|---|---|---|---|---|---|
| mean score, start | 2.6 | 1.8 | 3.0 | 2.0 | 1.9 | 2.0 | 2.3 | 1.5 | 1.9 |
| mean score, 4 wks | 2.6 | 2.0 | 3.1 | 2.1 | 1.9 | 2.1 | 2.4 | 2.1 | 1.9 |
| % improved | 13% | 13% | 38% | 25% | 13% | 50% | 38% | 50% | 13% |
| p values | 0.41 | 0.26 | 0.38 | 0.32 | 0.08 | 0.57 | 0.14 | 0.72 | 0.15 |

1^{a}Over the past 4 weeks how often did you experience discomfort during sexual intercourse? (1 = Almost always, 2 = Most times, 3 = Sometimes, 4 = A few times, 5 = Almost never).
2^{b}Over the past 4 weeks how often did you experience dryness during sexual intercourse? (1 = Almost always, 2 = Most times, 3 = Sometimes, 4 = A few times, 5 = Almost never).
3^{c}Over the past 4 weeks how often did you attempt sexual intercourse? (1 = 0, 2 = 1-2, 3 = 3-4, 4 = 5-6, 5 = 7-10, 6 = 11+).
4^{d}Over the past 4 weeks how often have you felt sexual desire? (1 = Almost never, 2 = A few times, 3 = Sometimes, 4 = Most times, 5 = Almost always).
5^{e}Over the past 4 weeks how would you rate your level of sexual desire? (1 = Very low, 2 = Low, 3 = Moderate, 4 = High, 5 = Very high).
6^{f}Over the past 4 weeks how satisfied have you been with your overall sex life? (1 = Very dissatisfied, 3 = About equally satisfied and dissatisfied, 5 = Very satisfied).
7^{g}Over the past 4 weeks how satisfied have you been with your sexual relationship with your partner? (1 = Very dissatisfied, 3 = About equally satisfied and dissatisfied, 5 = Very satisfied).
8^{h}Over the past 4 weeks, when you had sexual stimulation or intercourse, how often did you have the feeling of orgasm? (1 = Almost never, 2 = A few times, 3 = Sometimes, 4 = Most times, 5 = Almost always).
9ⁱOver the past 4 weeks, when you had sexual stimulation or intercourse, how would you rate your degree of clitoral sensation? (1 = Very low, 2 = Low, 3 = Moderate, 4 = High, 5 = Very high).

TABLE 2
Summary results from the Female Sexual Function Index (FSFI). Active vs. Placebo

| | | | | | | | | | |
|---|---|---|---|---|---|---|---|---|---|
| FSFI Question | 1^{a} | 2^{b} | 3^{c} | 4^{d} | 5^{e} | 6^{f} | 7^{g} | 8^{h} | 9ⁱ |

Active, total: 46 women

| | | | | | | | | | |
|---|---|---|---|---|---|---|---|---|---|
| mean score, start | 3.5 | 2.9 | 2.8 | 2.0 | 1.7 | 2.0 | 2.4 | 2.7 | 2.7 |
| mean score, 4 wks | 3.9 | 3.7 | 3.6 | 2.8 | 2.7 | 3.0 | 3.4 | 3.4 | 3.3 |
| % improved | 28% | 46% | 64% | 58% | 64% | 62% | 58% | 47% | 53% |

Placebo, total: 47 women

| | | | | | | | | | |
|---|---|---|---|---|---|---|---|---|---|
| mean score, start | 3.6 | 2.9 | 3.2 | 1.9 | 1.8 | 2.2 | 2.5 | 2.5 | 2.5 |
| mean score, 4 wks | 3.6 | 2.9 | 3.3 | 2.2 | 2.1 | 2.5 | 2.9 | 2.7 | 2.7 |
| % improved | 21% | 26% | 28% | 36% | 43% | 38% | 36% | 32% | 34% |
| p values | 0.16 | 0.02 | <0.001 | 0.03 | 0.008 | 0.009 | 0.02 | 0.06 | 0.04 |

Active, PRE + PERI only: 35 women

| | | | | | | | | | |
|---|---|---|---|---|---|---|---|---|---|
| mean score, start | 2.9 | 2.5 | 2.3 | 1.6 | 1.4 | 1.5 | 1.9 | 2.1 | 2.1 |
| mean score, 4 wks | 3.2 | 3.0 | 3.0 | 2.4 | 2.2 | 2.4 | 2.7 | 2.8 | 2.7 |
| % improved | 31% | 51% | 71% | 65% | 71% | 68% | 56% | 50% | 59% |

Placebo, PRE + PERI only: 39 women

| | | | | | | | | | |
|---|---|---|---|---|---|---|---|---|---|
| mean score, start | 3.1 | 2.6 | 2.7 | 1.6 | 1.5 | 1.9 | 2.1 | 2.2 | 2.2 |
| mean score, 4 wks | 3.1 | 2.5 | 2.8 | 1.8 | 1.8 | 2.2 | 2.5 | 2.4 | 2.4 |
| % improved | 23% | 28% | 26% | 38% | 49% | 36% | 36% | 28% | 38% |
| p values | 0.15 | 0.02 | <0.001 | 0.03 | 0.01 | 0.003 | 0.04 | 0.02 | 0.06 |

1^{a}Over the past 4 weeks how often did you experience discomfort during sexual intercourse? (1 = Almost always, 2 = Most times, 3 = Sometimes, 4 = A few times, 5 = Almost never).
2^{b}Over the past 4 weeks how often did you experience dryness during sexual intercourse? (1 = Almost always, 2 = Most times, 3 = Sometimes, 4 = A few times, 5 = Almost never).
3^{c}Over the past 4 weeks how often did you attempt sexual intercourse? (1 = 0, 2 = 1-2, 3 = 3-4, 4 = 5-6, 5 = 7-10, 6 = 11+).
4^{d}Over the past 4 weeks how often have you felt sexual desire? (1 = Almost never, 2 = A few times, 3 = Sometimes, 4 = Most times, 5 = Almost always).
5^{e}Over the past 4 weeks how would you rate your level of sexual desire? (1 = Very low, 2 = Low, 3 = Moderate, 4 = High, 5 = Very high).
6^{f}Over the past 4 weeks how satisfied have you been with your overall sex life? (1 = Very dissatisfied, 3 = About equally satisfied and dissatisfied, 5 = Very satisfied).
7^{g}Over the past 4 weeks how satisfied have you been with your sexual relationship with your partner? (1 = Very dissatisfied, 3 = About equally satisfied and dissatisfied, 5 = Very satisfied).
8^{h}Over the past 4 weeks, when you had sexual stimulation or intercourse, how often did you have the feeling of orgasm? (1 = Almost never, 2 = A few times, 3 = Sometimes, 4 = Most times, 5 = Almost always).
9ⁱOver the past 4 weeks, when you had sexual stimulation or intercourse, how would you rate your degree of clitoral sensation? (1 = Very low, 2 = Low, 3 = Moderate, 4 = High, 5 = Very high).

We claim:

1. A dietary supplement for wellness, comprising in a unit dosage a combination of (a) L-arginine, (b) ginseng, (c) ginkgo biloba, and (d) damiana.

2. The dietary supplement of claim 1, wherein said unit dosage is a capsule or a tablet.

3. The dietary supplement of claim 2 comprising
(a) about 50 mg to about 4500 mg L-arginine,
(b) about 0 mg to about 500 mg ginseng,
(c) about 0 mg to about 200 mg ginkgo biloba, and
(d) about 0 mg to about 300 mg damiana.

4. The dietary supplement of claim 3, further comprising in a unit dosage an amount of antioxidant vitamins, vitamin B complex, and minerals sufficient to provide up to 100% of the percent daily values of the antioxidant vitamins, vitamin B complex, and minerals upon administering an appropriate quantity of the unit dosages to achieve a desired daily dosage.

5. The dietary supplement of claim 4, comprising
(a) about 300 mg to about 2500 mg L-arginine,
(b) about 20 mg to about 200 mg ginseng,
(c) about 20 mg to about 70 mg ginkgo biloba, and
(d) about 5 mg to about 100 mg damiana.

6. The dietary supplement of claim 1, wherein the unit dosage is an aqueous composition.

7. The dietary supplement of claim 6, comprising
(a) about 500 mg to about 5000 mg L-arginine,
(b) about 25 mg to about 600 mg ginseng,
(c) about 12.5 mg to about 300 mg ginkgo biloba, and
(d) about 5 mg to about 100 mg damiana.

8. The dietary supplement of claim 7, further comprising in a unit dosage an amount of antioxidant vitamins, vitamin B complex, and minerals sufficient to provide up to 100% of the percent daily values of the antioxidant vitamins, vitamin B complex, and minerals upon administering an appropriate quantity of the unit dosages to achieve a desired daily dosage.

9. The dietary supplement of claim 8, wherein the daily dosage is in an amount of about 50 ml to about 250 ml.

10. The dietary supplement of claim 8, comprising
(a) about 750 mg to about 3000 mg L-arginine,
(b) about 100 mg to about 400 mg ginseng,
(c) about 50 mg to about 250 mg ginkgo biloba, and
(d) about 10 mg to about 80 mg damiana.

11. The dietary supplement of claim 10, wherein the dosage is an aqueous drink of about 100 ml comprising
(a) about 1500 mg L-arginine,
(b) about 100 mg Korean ginseng,
(c) about 100 mg ginkgo biloba,
(d) about 50 mg damiana,
(e) about 100% of the daily value of each of vitamins A, C, and E,
(f) about 100% of the daily value of each of thiamin, riboflavin, niacin, vitamin B-6, folate, vitamin B-12, biotin, pantothenic acid, and
(g) about 50% of the daily value of iron, calcium, and zinc.

12. The dietary supplement of claim 1, wherein said supplement is for women's wellness.

13. A method for enhancing libido, level of sexual desire, and/or frequency of sexual desire in a human subject, which method comprises administering a sufficient quantity of unit dosages of a dietary supplement to achieve a desired daily dosage, the unit dosage comprising (a) L-arginine, (b) Ginseng, (c) Ginkgo biloba, and (d) damiana.

14. The method of claim 13, wherein the administering a unit dosage comprises administering the dietary supplement as a capsule or a tablet.

15. The method of claim 13, wherein the administering a unit dosage comprises administering
(a) about 500 mg to about 3000 mg L-arginine,
(b) about 0 mg to about 300 mg ginseng,
(c) about 0 mg to about 150 mg ginkgo biloba, and
(d) about 0 mg to about 150 mg damiana.

16. The method of claim 15, wherein the administering the desired daily dosage of the dietary supplement comprises administering up to 100% of the percent daily values of antioxidant vitamins, vitamin B complex, and minerals.

17. The method of claim 16, wherein the administering the unit dosage comprises administering
(a) about 2000 mg to about 3000 mg L-arginine,
(b) about 50 mg to about 150 mg ginseng,
(c) about 20 mg to about 70 mg ginkgo biloba, and
(d) about 10 mg to about 100 mg damiana.

18. The method of claim 13, wherein the administering the dietary supplement comprises administering as an aqueous composition.

19. The method of claim 18, wherein the administering the unit dosage comprises administering
(a) about 500 mg to about 5000 mg L-arginine,
(b) about 25 mg to about 600 mg ginseng,
(c) about 12.5 mg to about 300 mg ginkgo biloba, and
(d) about 5 mg to about 300 mg damiana.

20. The method of claim 19, wherein the administering the desired daily dosage of the dietary supplement comprises administering up to 200% of the percent daily values of antioxidant vitamins and vitamin B complex, and up to 100% of the percent daily value of minerals.

21. The method of claim 20, wherein the administering the unit dosage comprises delivering as a beverage of about 50 ml to 600 ml.

22. The method of claim 13, wherein the administering comprises administering a combination daily to a subject for a time sufficient for sexually arousing the subject more easily than without administration of the combination.

23. The method of claim 13, wherein the administering comprises administering a combination to the subject for a time sufficient for improving the quality of orgasm for the subject.

24. The method of claim 13, wherein the dietary supplement is administered to the subject for a period of time sufficient to improve the frequency of desire to engage in sexual activity.

25. The method of claim 13, wherein the administering to the human subject comprises administering to a female subject.
